(19) United States
(12) Patent Application Publication
(10) Pub. No.: US 2025/0076221 A1
(43) Pub. Date: Mar. 6, 2025

(54) CORRECTING AMBIENT CAPILLARY PRESSURE CURVES TO ACCOUNT FOR DOWNHOLE RESERVOIR CONDITIONS

(71) Applicants: Schlumberger Technology Corporation, Sugar Land, TX (US); Saudi Arabian Oil Company, Dhahran (SA)

(72) Inventors: Mohammed Fadhel Al-Hamad, Dhahran (SA); Shouxiang Mark Ma, Dhahran (SA); Denis Vladimirovich Klemin, Houston, TX (US); Wael Abdallah, Dhahran (SA)

(21) Appl. No.: 18/457,717

(22) Filed: Aug. 29, 2023

(51) Int. Cl. G01N 23/046; G01N 1/40; G01N 23/083; G01N 33/24; G06F 30/20

(52) U.S. Cl. CPC G01N 23/046; G01N 1/4077; G01N 23/083; G01N 33/24; G06F 30/20

(57) ABSTRACT

Systems and methods are described for correcting ambient condition capillary pressure curves. In an example, the capillary pressure curve of a porous medium can be measured in non-reservoir conditions. Samples of the porous medium can be scanned at various confining pressures, and digital models of the scans can be created by a computing device. The computing device can simulate a porous plate experiment on the digital models to create a capillary pressure curve for each model. The computing device can calculate fitting equations for each capillary pressure curve according to capillary pressure points. The fitting equations can be averaged together and applied to the capillary pressure curve of the porous medium to correct for the non-reservoir conditions.

### BACKGROUND

Capillary pressure is the difference in pressure at an interface between two immiscible fluids. A capillary pressure curve relates the relationship between capillary pressure and saturation, which depends on the interfacial tension, pore size, and pore surface wettability. Capillary pressure curves are often refereed as capillary pressure data, or in short capillary pressure. Capillary pressure is important in interfacial sciences and petroleum geo-science because it is used to determine fluids distribution in an oil and gas reservoir. For example, capillary pressure in porous medium is a key parameter in assessing saturation height function, fluid distribution, its production or/and recovery. Capillary pressure is therefore a key input parameter for static and dynamic geological models. In the industries dealing with porous media, several conventional and acceptable laboratory techniques are available to measure capillary pressure, such as the porous plate (which is), mercury injection capillary pressure (“MICP”), and the most common used one, the centrifuge method, which is quick, non-destructive, and is done with fluids used closely mimicking those existing in the subsurface reservoirs, i.e., water and oil and/or gas. Porous plate tests are often used as a reference because it is considered the most accurate method for measuring capillary pressure curves. However, porous plate tests take an extremely long time to complete because the test uses a semi-permeable membrane, which only allows the wetting phase to pass through.

One problem with capillary pressure tests is that they are generally done at ambient laboratory conditions (such as laboratory temperature and pressure) not representing the reservoir conditions. Ambient laboratory conditions neglect effects of reservoir pressure on rock mechanics and stresses. Because of this, laboratory data requires correction for accurate reservoir characterization.

As a result, a need exists for a methodology to correct capillary pressure data measured at ambient conditions to reservoir conditions for more representative geoscience applications of capillary pressure.

### SUMMARY

Examples described herein include systems and methods for converting centrifuge-laboratory derived ambient capillary pressure data to reservoir conditions, counting the effect of reservoir stresses. In an example, the capillary pressure of a porous medium can be measured in ambient laboratory (i.e., non-reservoir conditions). For example, the porous medium can be placed in a centrifuge apparatus where its capillary pressure is measured. The same porous medium (or portions thereof) can be scanned under various confining stress levels to create digital models of the porous medium under each confining stress level.

A computing device can create a capillary pressure curve for each digital model by simulating a porous plate technique on each model. The computing device can insert data obtained from the simulations into a fitting equation that is a function of water saturation and confining stress level. The computing device can then calculate parameters of the fitting equation (such as slope and intercept of linear equations). In an example, the computing device can calculate a fitting equation for each capillary pressure point identified in the digital models. A capillary pressure point can refer to a specific location in the model at which the capillary pressure is being evaluated or observed. For example, pores in a porous medium can correspond to capillary pressure points at a specific saturation. The computing device can identify each pore and calculate a fitting equation for each pore.

In an example, the computing device can average the fitting equations together to obtain an average fitting equation. In another example, the average fitting equation can be a function of capillary pressure. The computing device can insert the measured capillary pressure into the fitting equation to obtain a capillary pressure curve of the porous medium that is corrected for reservoir conditions.

Both the foregoing general description and the following detailed description are exemplary and explanatory only and are not restrictive of the examples, as claimed.

### BRIEF DESCRIPTION OF THE DRAWINGS

FIG. 1 is a flowchart of an example method converting centrifuge-laboratory derived ambient capillary pressure data to reservoir conditions.

FIG. 2 is an example system for converting centrifuge-laboratory derived ambient capillary pressure data to reservoir conditions.

### DESCRIPTION OF THE EXAMPLES

Reference will now be made in detail to the present examples, including examples illustrated in the accompanying drawings. Wherever possible, the same reference numbers will be used throughout the drawings to refer to the same or like parts.

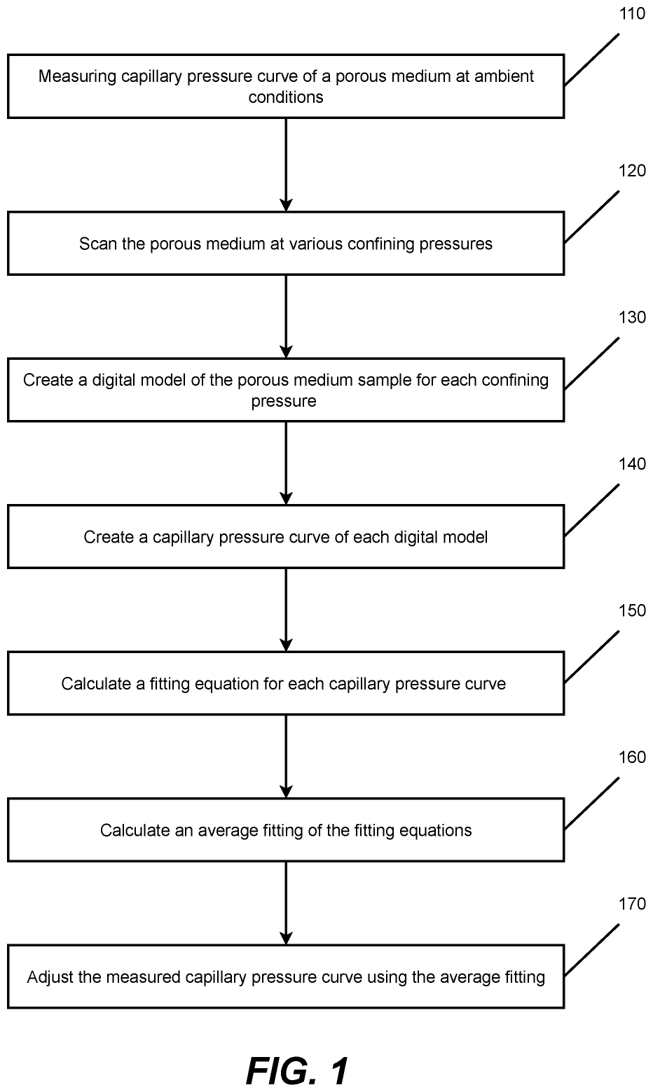

Systems and methods are described for correcting ambient capillary pressure curves. In an example, the capillary pressure curve of a porous medium can be measured in non-reservoir conditions. Samples of the porous medium can be scanned at various confining pressures, and digital models of the scans can be created by a computing device. The computing device can simulate a porous plate experiment on the digital models to create a capillary pressure curve for each model. The computing device can calculate fitting equations for each capillary pressure curve according to capillary pressure points. The fitting equations can be averaged together and applied to the capillary pressure of the porous medium to correct for the non-reservoir conditions. FIG. 1 is a flowchart of an example method for correcting ambient capillary pressure curves. At stage 110, the capillary pressure curve of a porous medium at ambient conditions can be measured. The porous medium can be a material or substance that contains interconnected void spaces or pores. These voids allow the medium to hold and transmit fluids, such as air, oil, or water. Examples of porous media include soil, rocks, sponges, filters, and certain types of polymers. The characteristics of a porous medium, such as pore size and connectivity, affect its ability to retain or release fluids and can play a significant role in various fields, including geology, hydrology, engineering, and biology.

Capillary pressure is the difference in pressure at the interface between two immiscible fluids. It is an important factor used to determine fluids distribution in a reservoir. The equilibrium oil reservoir capillary pressure can be calculated based on Equation 1 in Table 1 below, assuming a tube pore structure model:

TABLE 1
Equation 1
${P}_{C} = ( {P}_{o} - {P}_{w} ) = ( {ρ}_{w} - {ρ}_{o} ) gh = \frac{2 σcosθ}{{R}_{tube}}$

In Equation 1, P_{c} is the capillary pressure, P_{w} and Pₒ are the pressures in the water and oil phases, respectively, σ is the interfacial tension between oil and water, θ is the pore surface wettability in terms of contact angle, ρ_{w} and ρ_{O} are the water and oil densities, respectively, g acceleration due to gravity, h is the height of the fluid column above the free water level (“FWL”), which can be determined using formation pressure profiles. Such an equation relates P_{c} in a single pore diameter, which is not the case in reservoir rocks, where different pore geometries and sizes are connected by different pore throat sizes. However, a complex pore system can be approximated by a simple model of bundle of capillary tubes with radius of R_{tube}. The resulting equation shown in Equation 1 includes the dependence of the capillary pressure on the interfacial tension and pore surface wettability. These parameters are dynamic and are highly affected by the reservoir fluids composition.

The capillary pressure of the porous medium can be measured using any appropriate method. One such method is a centrifuge method. The centrifuge method involves subjecting a sample of the porous medium to centrifugal forces, which simulates the effect of gravity on fluid distribution within the material. For primary drainage capillary pressure curve measurement, the process can begin by saturating the porous sample with a wetting fluid, usually water. The sample is then placed in a centrifuge, with a chamber of oil as displacing fluid mimicking oil migration, and spun speeds varying from as low as possible to high simulating reservoir displacement pressure, generated from centrifugal forces. These forces cause the fluids within the sample to redistribute, and the resulting capillary pressure at a specific centrifuge speed versus fluid saturation relationship is measured. Imbibition of water displaces oil capillary pressure curves can also be generated using centrifuge with a slightly different set up and procedure. Examples of other methods that can be used to measure capillary pressure of the porous medium include mercury injection capillary pressure (“MICP”) and a porous plate technique.

The capillary pressure of the porous medium can be measured in ambient conditions. Ambient conditions refer to the surrounding environmental factors that typically exist in a specific location or setting. These conditions may include temperature, pressure, humidity, air composition, lighting, noise level, and other relevant physical parameters. As used herein, the term “ambient conditions” refers to conditions not representing pressure and temperature conditions in a reservoir, and therefore does to consider the actual formation mechanics and stresses. For example, ambient conditions can refer to conditions in a laboratory environment where capillary pressure of a porous medium may be measured.

At stage 120, the porous medium sample can be scanned at various confining pressures. The porous medium can be scanned using any available method. For example, the porous medium can be placed in a micro computed tomography (“Micro-CT”) scanner. A Micro-CT scanner is a 3-Dimensional (“3-D”) scanner that uses X-rays to view the inside of an object. The porous medium can be scanned at any number of confining pressures. For example, the porous medium can be scanned at 1,000, 3,000, 4,000, 5,000, and 6,000 pounds per square inch (“psi”).

In an example, different samples of the porous medium can be used for each confining pressure measurement. For example, mini-plugs can be extracted from the porous medium sample, and a different sister mini-plug can be scanned at each confining pressure in a Micro-CT scanning device. The mini-plugs can be of any predetermined size, such as 8 millimeters in diameter.

At stage 130, a computing device can create a digital model of the porous medium sample for each confining pressure. For example, the scan data can be inputted into modeling software running on the computing device. The scan data can include any data used to scan the porous medium, such as Micro-CT datasets. The modeling software can process the scan data to produce a digital model of the porous medium at each scanned confining pressure. In one example, the modeling software can process the additionally obtained scanning electron microscopy (“SEM”) imaging data. The digital model is constructed using rock imaging data that captures both resolved open pores and submicron pores in the porous medium sample.

At stage 140, the computing device can create a capillary pressure curve for each digital model. For example, the computing device can simulate the application of a porous plate technique to each of the digital models. A porous plate technique is a method for desaturating a core sample by placing one end in capillary contact with a porous plate and applying gas or oil under pressure to the remaining exposed surfaces of the testing sample. Only the liquid in the original fully saturated sample (such as water) is expelled through the porous plate. At different pressure stages, the produced fluid (such as water) is measured. At each pressure, desaturation continues until no more fluid production is observed, which can be very time consuming. With increased pressure, if little fluid can be produced, this condition is often called that the sample is at irreducible water saturation. Core samples can be desaturated to measure, for example, capillary pressure, irreducible water saturation, resistivity index, or nuclear magnetic resonance response. The computing device can use the data from the porous plate technique simulation to create an equation representing capillary pressure curve for each digital model. The capillary pressure curves can represent the capillary pressure as a function of the measured water saturation.

At stage 150, the computing device can calculate a fitting equation for each sample at a constant capillary pressure. The fitting equation can represent the water saturation as a function of the confining pressure. For example, using the generated capillary pressure curves, the computing device can create fitting equations in the format of Equation 2 in Table 2.

TABLE 2
Equation 2

Sw = a (P_{cf}) + b

In Equation 2, Sw represents the calculated water saturation at a specified capillary pressure (Pc) and a given confining pressure (P_{cf}), a is a parameter representing the slope of the fitting equation, and b is a parameter representing the intercept of the fitting equation. The computing device can input the measured water saturation (Sw) for each P_{cf} and perform a regression analysis to calculate the a and b parameters. In an example, the a and b parameters can be calculated as a function of Pc. As an example, the a parameter can be 0.004*(P_{c}^{-0.203}) and the b parameter can be 61.301*(P_{c}^{-0.196}).

In an example, the a and b parameters can be calculated for each capillary pressure (Pc) point identified in the modeled porous medium. A capillary pressure point can refer to a specific location in the model at which the capillary pressure is being evaluated or observed. For example, pores in a porous medium can correspond to capillary pressure points. The computing device can identify each pore and calculate a fitting equation for each pore.

At stage 160, the computing device can calculate an average fitting of the fitting equations. For example, the computing device can average the a and b parameters for each capillary pressure point to obtain an average fitting equation representative of the entire porous medium.

At stage 170, the computing device can adjust the measured capillary pressure curve using the average fitting equation. For example, the computing device can input a and b values into Equation 2, at a specific Pc. The computing device can then input a confining pressure (P_{cf}) to calculate a corrected water saturation (Sw) value at the corresponding confining pressure level, at that specific Pc.

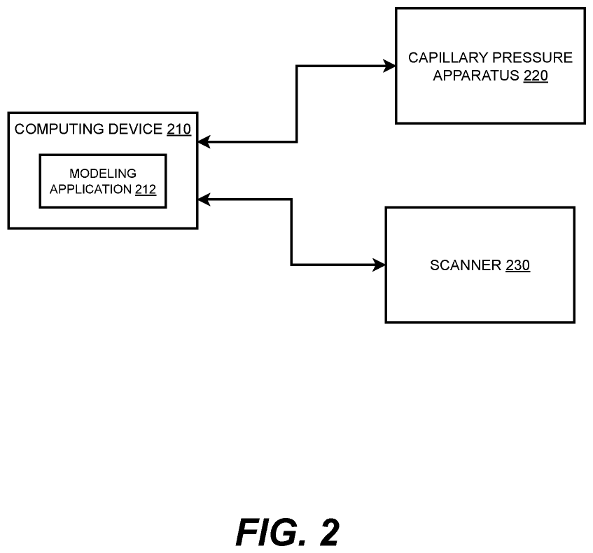

FIG. 2 is an example system for correcting ambient capillary pressure curves. The example system can include a computing device 210 with a modeling application 212. The computing device 210 can be one or more processor-based devices, such as a personal computer, tablet, or cell phone. The modeling application 212 can be a software application that includes functionality for constructing digital models of a porous medium. In an example, the modeling application 212 can create digital models of a porous medium using data from a scanner 230. The scanner 230 can be any device that can scan a porous medium. For example, the scanner 230 can be a Micro-CT scanner, which is a 3-D scanner that uses X-rays to view the inside of an object.

In an example, a user can place a porous medium, such as a rock from a reservoir, into the scanner 230. The scanner 230 can scan the porous medium and transfer the scan data to the computing device 210. The scanner 230 can transfer the scan data using any appropriate communication protocol. For example, the scanner 230 can transfer the data through a hardwire connection between the scanner 230 and the computing device 210. Alternatively, the scanner 230 can transfer the scan data wirelessly, such as through BLUETOOTH or WIFI. In another example, the scanner 230 can upload the scan data to the server, the modeling application 212 can access the scan data at the server.

A capillary pressure apparatus 220 can also send data to the computing device 210. The capillary pressure apparatus 220 can be any apparatus that can measure capillary pressure in a porous medium. For example, the capillary pressure apparatus 220 can be a centrifuge that subjects a sample of porous medium to centrifugal forces, which simulates the effect of gravity on fluid distribution within the material. The capillary pressure apparatus 220 can transfer data using any appropriate communication protocol, such as a through a wired or wireless connection or through a server.

The modeling application 212 can process data received from the scanner 230 to correct capillary pressure reported by the capillary pressure apparatus 220. For example, as described herein, the modeling application 212 can create a digital model of a porous medium scanned by the scanner 230. The modeling application 212 can create capillary pressure curves of the digital model based on a modeled porous plate technique. This technique is done by numerical modeling at pore scale using multiphase hydrodynamic simulator. The modeling application 212 can then create fitting equations using the capillary pressure curves and apply the fitting equations to the data received from the capillary pressure apparatus 220.

Other examples of the disclosure will be apparent to those skilled in the art from consideration of the specification and practice of the examples disclosed herein. Though some of the described methods have been presented as a series of steps, it should be appreciated that one or more steps can occur simultaneously, in an overlapping fashion, or in a different order. The order of steps presented are only illustrative of the possibilities and those steps can be executed or performed in any suitable fashion. Moreover, the various features of the examples described here are not mutually exclusive. Rather any feature of any example described here can be incorporated into any other suitable example. It is intended that the specification and examples be considered as exemplary only, with a true scope and spirit of the disclosure being indicated by the following claims.

What is claimed is:

1. A method for correcting ambient capillary pressure curves, comprising:
measuring capillary pressure curve of a porous medium in non-reservoir conditions;
scanning the porous medium at a plurality of confining pressures;
creating a digital model of the porous medium at each of the plurality of confining pressures;
creating a capillary pressure curve for each digital model;
calculating a fitting equation for each capillary pressure curve;
calculating an average fitting equation based on the fitting equations; and
adjusting the measured capillary pressure curve using the average fitting equation.

2. The method of claim 1, wherein the capillary pressure curve of the porous medium is measured in a centrifuge apparatus.

3. The method of claim 1, wherein the porous medium is scanned using a micro computed tomography scanner.

4. The method of claim 1, wherein scanning the porous medium at a plurality of confining pressures comprises:
extracting a plurality of micro plugs from the porous medium; and
scanning each of the plurality of micro plugs at a different confining pressure.

5. The method of claim 1, wherein creating the capillary pressure curve for each digital model includes simulating a porous plate technique for each of each digital model.

6. The method of claim 1, wherein the fitting equation for each capillary pressure curve is in a form that calculates the water saturation as a function of the confining pressure, at a specific capillary pressure, and wherein calculating the fitting equation for each capillary pressure curve includes inserting the water saturation for each confining pressure and performing a regression analysis to obtain parameter values corresponding to slope and intercept.

7. The method of claim 6, wherein the parameter values are functions of the capillary pressure.

8. A non-transitory, computer-readable medium containing instructions that, when executed by a hardware-based processor, causes the processor to perform stages for correcting ambient capillary pressure curves, the stages comprising:
measuring capillary pressure curve of a porous medium in non-reservoir conditions;
scanning the porous medium at a plurality of confining pressures;
creating a digital model of the porous medium at each of the plurality of confining pressures;
creating a capillary pressure curve for each digital model;
calculating a fitting equation for each capillary pressure curve;
calculating an average fitting equation based on the fitting equations; and
adjusting the measured capillary pressure curve using the average fitting equation.

9. The non-transitory, computer-readable medium of claim 8, wherein the capillary pressure curve of the porous medium is measured in a centrifuge apparatus.

10. The non-transitory, computer-readable medium of claim 8, wherein the porous medium is scanned using a micro computed tomography scanner.

11. The non-transitory, computer-readable medium of claim 8, wherein scanning the porous medium at a plurality of confining pressures comprises:
extracting a plurality of micro plugs from the porous medium; and
scanning each of the plurality of micro plugs at a different confining pressure.

12. The non-transitory, computer-readable medium of claim 8, wherein creating the capillary pressure curve for each digital model includes simulating a porous plate technique for each of each digital model.

13. The non-transitory, computer-readable medium of claim 8, wherein the fitting equation for each capillary pressure curve is in a form that calculates the water saturation as a function of the confining pressure, at a specific capillary pressure, and wherein calculating the fitting equation for each capillary pressure curve includes inserting the water saturation for each confining pressure and performing a regression analysis to obtain parameter values corresponding to slope and intercept.

14. The non-transitory, computer-readable medium of claim 13, wherein the parameter values are functions of the capillary pressure.

15. A system for correcting ambient capillary pressure curves, comprising:
a capillary pressure apparatus;
a scanning device;
a memory storage including a non-transitory, computer-readable medium comprising instructions; and
a hardware-based processor that executes the instructions to carry out stages comprising:
receiving, from the capillary pressure apparatus, a measurement of capillary pressure curve of a porous medium in non-reservoir conditions;
receiving, from the scanning device, scans of the porous medium at a plurality of confining pressures;
creating a digital model of the porous medium at each of the plurality of confining pressures;
creating a capillary pressure curve for each digital model;
calculating a fitting equation for each capillary pressure curve;
calculating an average fitting equation based on the fitting equations; and
adjusting the measured capillary pressure curve using the average fitting equation.

16. The system of claim 15, wherein the capillary pressure apparatus is a centrifuge apparatus.

17. The system of claim 15, wherein the scanning device is a micro computed tomography scanner.

18. The system of claim 15, wherein each received scan of the porous medium is a scan of a micro plug extracted from the porous medium sample, each micro plug having been scanned at a different confine pressure.

19. The system of claim 15, wherein creating the capillary pressure curve for each digital model includes simulating a porous plate technique for each digital model.

20. The system of claim 15, wherein the fitting equation for each capillary pressure curve is in a form that calculates the water saturation as a function of the confining pressure, and wherein calculating the fitting equation for each capillary pressure curve includes inserting the water saturation for each confining pressure and performing a regression analysis to obtain parameter values corresponding to slope and intercept.
